(19) United States
(12) Patent Application Publication
(10) Pub. No.: US 2026/0207750 A1
(43) Pub. Date: Jul. 23, 2026

(54) THE USE OF HYDROXYPROPYL-BETA-CYCLODEXTRIN IN STABILIZING ANTIBODY-DRUG CONJUGATE FORMULATIONS

(71) Applicant: WUXI BIOLOGICS IRELAND LIMITED, Dundalk, Co Louth (IE)

(72) Inventors: Yunyun WANG, Shanghai (CN); Weipeng MAO, Shanghai (CN); Tong DAI, Shanghai (CN); Quanmin CHEN, Shanghai (CN); Zheming GUO, Shanghai (CN)

(21) Appl. No.: 19/144,369

(22) PCT Filed: Dec. 28, 2023
(86) PCT No.: PCT/CN2023/142828

§ 371 Date: Jun. 27, 2025

(30) Foreign Application Priority Data
Dec. 30, 2022 (CN) 202211727542.X

(51) Int. Cl. A61K 47/40; A61K 9/08; A61K 47/18; A61K 47/26; A61K 47/68

(52) U.S. Cl. CPC A61K 47/40; A61K 9/08; A61K 47/183; A61K 47/26; A61K 47/68031; A61K 47/68037; A61K 47/6809

(57) ABSTRACT

Provided herein is a composition for stabilizing antibody-drug conjugate (ADC) formulations, characterized in that the composition comprises: hydroxypropyl-β-cyclodextrin of about 2% to 16% (w/v), optionally 0-10% (w/v) saccharides or polyols; surfactants; and buffering solution; wherein the pH of the composition is in the range of 4.5-8.0, preferably 5.5-6.5 or 5.7-6.3. Also provided is the use of hydroxypropyl-β-cyclodextrin for stabilizing antibody-drug conjugate (ADC) formulation.

### (1) TECHNICAL FIELD

The present invention belongs to the field of biopharmaceuticals, particularly in the production of antibody-drug conjugates (ADCs), and relates to formulations that provide additional stability for ADCs.

### (2) BACKGROUND TECHNOLOGY

Antibody-Drug Conjugates (ADCs) are a hybrid bio-therapeutics composed of site-specific antibody and chemotherapeutic agents. Owing to the selectivity of antibodies, ADCs can deliver cytotoxic agents to the tumor sites which are extensively surveyed in clinical investigations. Due to the chemico-physical nature of protein itself, payload property and conjugation methods, the incorporation of chemo-drugs to antibodies usually lead to decrease in protein conformational stability, increase in molecular hydrophobicity and aggregation propensity. (Adem, Y. T., Schwarz, K. A., Duenas, E., Patapoff, T. W., Galush, W. J., &Esue, O. (2014); Ross, P. L., &Wolfe, J. L. (2016). Physical and Chemical Stability of Antibody Drug Conjugates: Current Status. Journal of Pharmaceutical Sciences, 105 (2), 391-397). Regarding the regulation concerns on immunogenicity caused by protein aggregates, it is critical to control ADC aggregation formation during its Chemistry Manufacturing and Control (CMC) development. The above factors pose significant challenges to the development of formulations of ADC macromolecular drugs. For the stability of formulation products, as of now, most of the antibody-drug conjugate drug products that have been launched globally have adopted conservative and high-cost lyophilized formulation processes.

For example, monomethyl auristatin E (MMAE) is widely used in the development of antibody-drug conjugates. As reported, conjugating MMAE to antibodies leads to higher overall hydrophobicity of ADCs, resulting in stronger protein-protein interactions. This further facilitates the formation of aggregates in MMAE class ADCs that conjugated via interchain disulfide bonds during manufacturing and storage. The aggregation of protein drugs will affect the shelf life, pharmacokinetic performance, etc., and is a critical quality attribution highly concerned by administration institutes.

Therefore, there is still a need to find suitable stable systems for ADC formulations.

### (3) SUMMARY OF THE INVENTION

Therefore, one object of the present invention is to provide a composition for stabilizing antibody-drug conjugate (ADC) formulations, characterized in that the composition comprises:
hydroxypropyl-β-cyclodextrin of about 2% to 16% (w/v), preferably of about 2%, 4%, or 8%, more preferably 8% (w/v);
optionally 0-10% (w/v) saccharides or polyols, preferably sucrose;
surfactants; and
buffering solution;
wherein the pH of the composition is in the range of 4.5-8.0, preferably 5.5-6.5 or 5.7-6.3, most preferably, 5.5 or 6.0.

In one embodiment of this aspect, the composition comprises 0-8% (w/v), preferably 4% (w/v) sucrose, and more preferably no sucrose.

In another embodiment of the present aspect of the invention, the composition comprises 8% (w/v) hydroxypropyl-β-cyclodextrin and 0% (w/v) sucrose.

In a further embodiment of the present aspect of the invention, the composition further comprises 20 mM histidine, and wherein the pH of the composition is 6.0; wherein the surfactant is preferably polysorbate 80, preferably of about 0.02% (w/v).

In another preferred embodiment of the present aspect of the invention, the drug portion of the antibody-drug conjugate (ADC) comprises a drug selected from the group consisted of Monomethyl auristatin E (MMAE), Doxorubicin, or SN-38, preferably a drug selected from MC-Val-Cit-PAB-MMAE, CL2A-SN38 (SN-38 connected by CL2A linker), or Doxorubicin-succinimidyl 4-[N-maleimidomethyl]cyclohexane-1-carboxylate (SMCC), and wherein the antibody portion of ADC is preferably a monoclonal antibody (mAb), more preferably selected from the group consisted of monoclonal antibodies targeting against tumor necrosis factor α (TNF-α), monoclonal antibodies targeting against Vascular Endothelial Growth Factor (VEGF), monoclonal antibodies targeting against Cluster of Differentiation 20 (CD20), or monoclonal antibodies targeting against Human Epidermal Growth Factor Receptor 2 (HER2); more preferably the antibody portion is IgG isotype, even more preferably, the IgG1 or IgG4 isotype. In a preferred embodiment, the drug: antibody ratio (DAR) of the ADC is about 8:1 to 1:1, with preferred values of 1, 2, 3, 4, 5, 6, 7, and 8, more preferable that DAR can be selected from 2, 4, or 7.

In another aspect of the invention, it discloses the use of hydroxypropyl-β-cyclodext rin for stabilizing antibody-drug conjugate (ADC) formulation, characterized in that the formulation comprises Hydroxypropyl-β-cyclodextrin of about 2% to 16% (w/v), preferably of about 2%, 4%, or 8%, more preferably 8% (w/v);
optionally 0-10% (w/v) saccharides or polyols, preferably sucrose;
surfactants; and
buffering solution;
wherein the pH of the composition is in the range of 4.5-8.0, preferably 5.5-6.5 or 5.7-6.3; most preferably 5.5 or 6.0.

In a preferred embodiment of the present aspect of the invention, wherein the formulation comprises antibody-drug conjugate, wherein the drug portion of the antibody-drug conjugate (ADC) comprises a drug selected from the group consisted of Monomethyl auristatin E (MMAE), Doxorubicin, or SN-38, preferably a drug selected from MC-Val-Cit-PAB-MMAE, CL2A-SN38 (SN-38 connected by CL2A linker), or Doxorubicin-succinimidyl 4-[N-maleimidomethyl]cyclohexane-1-carboxylate (SMCC), and wherein the antibody portion of ADC is preferably a monoclonal antibody (mAb), more preferably selected from the group consisted of monoclonal antibodies targeting against tumor necrosis factor α (TNF-α), monoclonal antibodies targeting against Vascular Endothelial Growth Factor (VEGF), monoclonal antibodies targeting against Cluster of Differentiation 20 (CD20), or monoclonal antibodies targeting against Human Epidermal Growth Factor Receptor 2 (HER2); more preferably is IgG isotype, even more preferably, the IgG1 or IgG4 isotype, preferably, wherein the formulation comprises 8% (w/v) hydroxypropyl-β-cyclod extrin and 0% (w/v) sucrose, more preferably the formulation further comprises 20 mM histidine, and wherein the pH of the composition is 6.0; wherein the surfactant is polysorbate 80 and preferably of about 0.02% (w/v). In a preferred embodiment, the drug: antibody ratio (DAR) of the ADC is about 8:1 to 1:1, with preferred values of 1, 2, 3, 4, 5, 6, 7, and 8, more preferable that DAR can be selected from 2, 4, or 7.

In a preferred embodiment of the present aspect of the invention, the Monomethyl auristatin E (MMAE) is MC-Val-Cit-PAB-MMAE, of following formula:

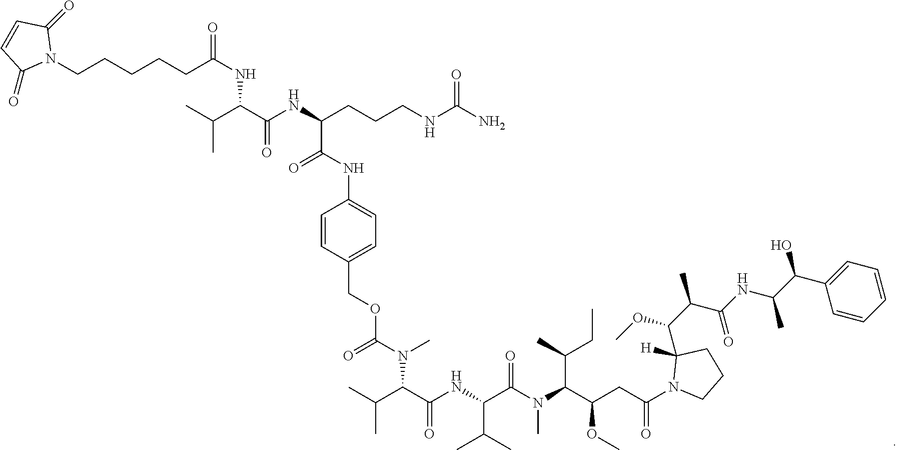 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

In another preferred embodiment, the antibody is an IgG isotype, preferably IgG1 or IgG4; more preferably, the antibody is conjugated with MMAE via interchain cysteine; wherein the preferred MMAE: antibody ratio is between 8:1 and 1:1, with preferred values of 1, 2, 3, 4, 5, 6, 7, and 8, more preferable that DAR can be selected from 2, 4, or 7.

In a further preferred embodiment, wherein the formulation comprises 2 mg/ml to 20 mg/ml of ADC, preferably 10 mg/ml of ADC.

In another preferred embodiment, wherein the SMCC is Doxorubicin-succinimidyl 4-[N-maleimidomethyl]cyclohexane-1-carboxylate (SMCC), with the following formula:

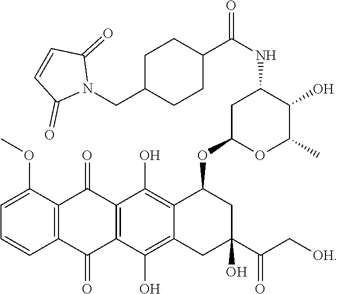 [SMILES: COc1cccc2c1C(=O)c1c(O)c3c(c(O)c1C2=O)C[C@@](O)(C(=O)CO)C[C@@H]3O[C@H]1C[C@H](NC(=O)C2CCC(CN3C(=O)C=CC3=O)CC2)[C@H](O)[C@H](C)O1]

In a preferred embodiment, the antibody is an IgG isotype, preferably IgG1 or IgG4; more preferably, the antibody is conjugated with SMCC via interchain cysteine; wherein the preferred SMCC: antibody ratio is between 8:1 and 1:1, with preferred values of 1, 2, 3, 4, 5, 6, 7, and 8, more preferable that DAR can be selected from 2, 4, or 7. In a more preferred embodiment, the formulation comprises 2 mg/ml to 20 mg/ml of ADC, preferably 10 mg/ml of ADC

In another preferred embodiment, the SN-38 is CL2A-SN38 (SN-38 linked by CL2A linker), having the following formula:

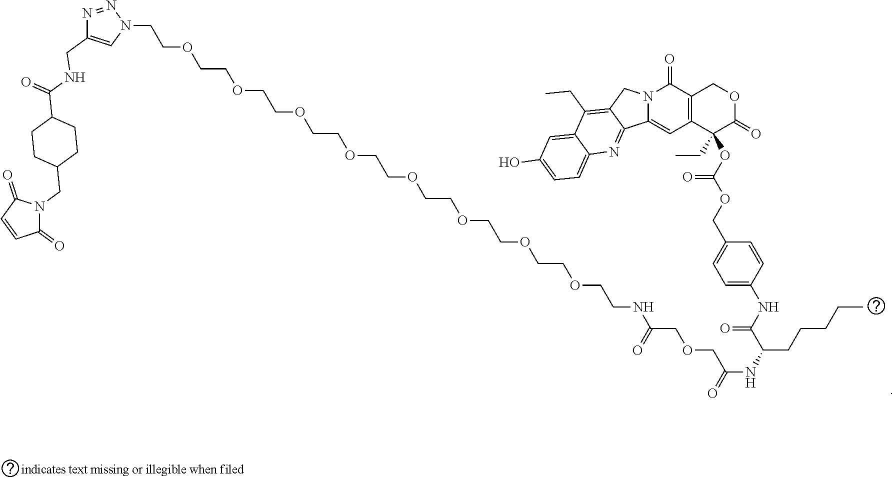 [SMILES: CCCCC[C@H](NC(=O)COCC(=O)NCCOCCOCCOCCOCCOCCOCCOCCOCCn1cc(CNC(=O)C2CCC(CN3C(=O)C=CC3=O)CC2)nn1)C(=O)Nc1ccc(COC(=O)O[C@]2(CC)C(=O)OCc3c2cc2n(c3=O)Cc3c-2nc2ccc(O)cc2c3CC)cc1]

In a preferred embodiment, the antibody is an IgG isotype, preferably IgG1 or IgG4; more preferably, the antibody is conjugated with SN38 via interchain cysteine; wherein the preferred SN38:antibody ratio is between 8:1 and 1:1, with preferred values of 1, 2, 3, 4, 5, 6, 7, and 8, more preferable that DAR can be selected from 2, 4, or 7. In one preferred embodiment, the formulation comprises 2 mg/ml to 20 mg/ml of ADC, preferably 10 mg/ml of ADC.

The advantage of the present invention is that by adding hydroxypropyl-β-cyclodextrin (HPBCD) to the ADC, the formulation brought about the following advantages:
1. substantial lower high molecular species increase at thermal stress (40° C.). This finding is demonstrated in ADCs with 3 types of linker payload (MC-VC-PAB-MMAE or CL2A-SN38 or Doxorubicin-SMCC), 5 antibodies (TNF-α or VEGF or CD20 or HER2 targeted and 1 general control antibody) and multiple Drug to antibody ratio (DAR) ranging from 2-7.
2. substantial lower high molecular species increase at accelerated thermal condition (25° C.)
3. substantial lower high molecular species increase after agitation (300 rpm, 25° C.)
4. substantial lower high molecular species increase after light illumination and ultraviolet (UV) treatment (5000 Lux 10 days+UV 50 hours), therefore obtaining formulation with significant stability.

The other features and advantages of various embodiments will be specifically described in the following disclosure, and partially be obvious or understood based on the specification or by implementing various embodiments. The objects and other advantages of various embodiments will be realized or reached through, in particular, the elements and combinations thereof disclosed in the present specification and claims.

Unless otherwise specified, the reagents, cells, and instrument devices used in the present disclosure are all commercially and commonly available to the public.

### (4) DESCRIPTION OF THE FIGURES

FIG. 1 illustrated trend of SEC-HPLC high molecular species change of 3 formulations of ADC-A (DAR4) at 2 mg/ml (Formulations 1-3), incubation at 40° C. for 14 and 28 days.

FIG. 2 illustrated trend of SEC-HPLC high molecular species change of 3 formulations of ADC-B (DAR4) at 2 mg/ml (Formulations 4-6), incubation at 40° C. for 14 and 28 days.

FIG. 3 Illustrated trend of SEC-HPLC high molecular species change of 3 formulations of ADC-C (DAR4) at 2 mg/ml (Formulations 7-9), incubation at 40° C. for 14 and 28 days.

FIG. 4 Illustrated trend of SEC-HPLC high molecular species change of 5 formulations of ADC-C (DAR4) at 10 mg/ml (Formulations 10-14), incubation at 40° C. for 7, 14 and 28 days.

FIG. 5 Illustrated trend of SEC-HPLC high molecular species change of 3 formulations of ADC-C (DAR2) at 10 mg/ml (Formulations 15-17), incubation with shake (300 rpm, 25° C.) for 3 and 7 days.

FIG. 6 Illustrated trend of SEC-HPLC high molecular species change of 3 formulations of ADC-C (DAR4) at 10 mg/ml (Formulations 18-20), incubation with shake (300 rpm, 25° C.) for 3 and 7 days.

FIG. 7 Illustrated trend of SEC-HPLC high molecular species change of 3 formulations of ADC-C (DAR7) at 10 mg/ml (Formulations 21-23), incubation with shake (300 rpm, 25° C.) for 3 and 7 days.

FIG. 8 Illustrated trend of SEC-HPLC high molecular species change of 3 formulations of ADC-C (DAR2) at 10 mg/ml (Formulations 15-17), incubation at high temperature (40° C.) for 14 and 28 days.

FIG. 9 Illustrated trend of SEC-HPLC high molecular species change of 3 formulations of ADC-C (DAR4) at 10 mg/ml (Formulations 18-20), incubation at high temperature (40° C.) for 14 and 28 days.

FIG. 10 Illustrated trend of SEC-HPLC high molecular species change of 3 formulations of ADC-C (DAR7) at 10 mg/ml (Formulations 21-23), incubation at high temperature (40° C.) for 14 and 28 days.

FIG. 11 Illustrated trend of SEC-HPLC high molecular species change of 3 formulations of ADC-C (DAR2) at 10 mg/ml (Formulations 15-17), incubation at accelerated condition (25° C.) for 4, 12 and 24 weeks.

FIG. 12 Illustrated trend of SEC-HPLC high molecular species change of 3 formulations of ADC-C (DAR4) at 10 mg/ml (Formulations 18-20), incubation at accelerated condition (25° C.) for 4, 12 and 24 weeks.

FIG. 13 Illustrated trend of ADC-C (DAR7) (Formulations 21-23) SEC-HPLC high molecular species change at T0, 25° C. incubation for 4, 12 and 24 weeks.

FIG. 14 Illustrated trend of ADC-D (DAR6) (Formulations 24-25) SEC-HPLC high molecular species change at T0, 40° C. incubation for 2 and 4 weeks, 40° C. incubation for 4 weeks followed by 2 weeks 50° C. incubation.

FIG. 15 Illustrated trend of ADC-D (DAR2) (Formulations 26-27) SEC-HPLC high molecular species change at T0, 40° C. incubation for 2 and 4 weeks, 40° C. incubation for 4 weeks followed by 2 weeks 50° C. incubation.

FIG. 16 Illustrated trend of ADC-E (DAR6) (Formulations 28-29) SEC-HPLC high molecular species change at T0, 40° C. incubation for 2 and 4 weeks, 40° C. incubation for 4 weeks followed by 2 weeks 50° C. incubation.

FIG. 17 Illustrated trend of ADC-E (DAR2) (Formulations 30-31) SEC-HPLC high molecular species change at T0, 40° C. incubation for 2 and 4 weeks, 40° C. incubation for 4 weeks followed by 2 weeks 50° C. incubation.

FIG. 18 Illustrated trend of ADC-A (DAR6) (Formulations 32-33) SEC-HPLC high molecular species change at T0, 40° C. incubation for 2 and 4 weeks, 40° C. incubation for 4 weeks followed by 2 weeks 50° C. incubation.

FIG. 19 Illustrated trend of ADC-A (DAR2) (Formulations 34-35) SEC-HPLC high molecular species change at T0, 40° C. incubation for 2 and 4 weeks, 40° C. incubation for 4 weeks followed by 2 weeks 50° C. incubation.

FIG. 20 Illustrated trend of ADC-F (DAR2) (Formulations 36-37) SEC-HPLC high molecular species change at T0, 40° C. incubation for 2 and 4 weeks.

FIG. 21 Illustrated trend of ADC-G (DAR2) (Formulations 38-39) SEC-HPLC high molecular species change at T0, 40° C. incubation for 2 and 4 weeks.

FIG. 22 Illustrated trend of ADC-H (DAR2) (Formulations 40-41) SEC-HPLC high molecular species change at T0, 40° C. incubation for 2 and 4 weeks.

FIG. 23 Illustrated trend of ADC-I (DAR2) (Formulations 42-43) SEC-HPLC high molecular species change at T0, 40° C. incubation for 2 and 4 weeks.

FIG. 24 Illustrated trend of ADC-E (DAR6), ADC-E (DAR2), ADC-G (DAR2), ADC-I (DAR2) SEC-HPLC high molecular species change at T0, 5000 Lux white light illumination for 10 days, 5000 Lux white light illumination for 10 days followed by UV light illumination for 50 hours.

FIG. 25 Illustrated trend of ADC-A (DAR6) and ADC-A (DAR2) SEC-HPLC high molecular species change at T0, 5000 Lux white light illumination for 10 days, 5000 Lux white light illumination for 10 days followed by UV light illumination for 50 hours.

FIG. 26 Illustrated trend of ADC-F (DAR2) and ADC-H (DAR2) SEC-HPLC high molecular species change at T0, 5000 Lux white light illumination for 10 days, 5000 Lux white light illumination for 10 days followed by UV light illumination for 50 hours.

### (5) DETAILED DESCRIPTION

Hereinafter detailed description of some embodiments of the present invention will be provided, examples of which are illustrated in the accompanying figures. Although the present invention is described in conjunction with illustrated embodiments, it should be understood that they are not intended to limit the present invention to those embodiments. On the contrary, the present invention is intended to cover all substitutions, modifications, and equivalents that are defined by the accompanying claims.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

In the present disclosure, when the length of a time period, duration, or interval is expressed in “day (s)”, and a time point is expressed in “day (s)”, it means that time or timing is calculated or recognized in days, where numbers may not accurately represent multiples of 24 hours.

The term “antibody” or “antibody protein” used herein refers to immunoglobulins that are generated by reaction of body to the presence of antigens and bind to antigens, as well as antigen-binding fragments and genetically engineered variants thereof. Therefore, the term “antibody” includes, for example, complete monoclonal antibodies (such as antibodies generated using hybridoma technology) and antigen-binding antibody fragments, such as F(ab′)₂ and Fab fragments. This also includes genetically engineered complete antibodies and fragments, such as chimeric antibodies, humanized antibodies, single chain Fv fragments (scFvs), single chain antibodies, bifunctional antibodies, microbodies, linear antibodies, multivalent or multi-specific (such as bispecific) hybrid antibodies, etc. Therefore, the term “antibody” is generally referred to any protein that comprises antigen-binding site of antibody and can specifically bind to targeted antigen. The term “antibody” also includes antibodies themselves (“naked antibodies”) or antibodies that bind to cell growth inhibitors or cytotoxic agents. The antibodies provided herein can be any type of immunoglobulin molecule (e.g. IgG, IgE, IgM, IgD, and IgA) or any isotype (e.g., IgG1, IgG2, IgG3, IgG4, IgAQ1, and IgA2). In one embodiment, the antibody conjugated with MMAE is IgG1 or IgG4 isotype, more preferably IgG1 isotype.

The antibodies used herein target against TNF-α, VEGF or CD20 (differentiation cluster 20) or human epidermal growth factor receptor 2 (HER2), etc., and can target at tissue or cells related to diseases, such as cancer, to specifically realize their effects. These commercially available antibodies include, but not limited to Adalimumab (trade name: Humrira®) targeting against TNF-α; Bevacizumab (trade name: Avastin®) targeting VEGF; Rituximab (trade name: Rituxan®) targeting against CD20; Trastuzumab targeting Her2 (trade name: Herceptit®).

In a particular embodiment of this aspect, the formulation preferably comprises antibody-drug conjugate comprising Monomethyl auristatin E (MMAE), but it may also be selected from or not limited to auristatin or other derivatives thereof. The Monomethyl auristatin E (MMAE) is MC-Val-Cit-PAB-MMAE, which has the following formula:

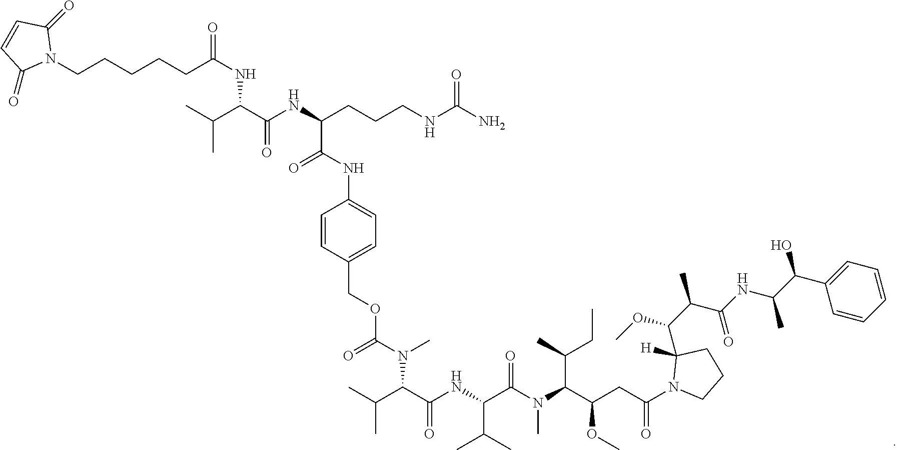 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

More preferably, the ADC formulation comprising MC-Val-Cit-PAB-MMAE and IgG1 linked via cysteine, wherein the formulation comprises 8% (w/v) hydroxypropyl-β-cyclodextrin, wherein the formulation does not comprise sucrose, and the formulation comprises 0.02% (w/v) polysorbate 80 and 20 mM histidine buffer at pH 6.0.

In a preferred embodiment, MMAE is preferably connected to antibody moiety via linker unit, and the preferred DAR (drug:antibody ratio) can be between 8:1 and 1:1, preferably 1, 2, 3, 4, 5, 6, 7, or 8. In the preferred embodiments of the present invention, DAR can be selected from 2, 4, or 7.

In a preferred embodiment, MMAE class ADCs can be prepared by conjugating antibody unit (such as preferred IgG1 antibodies) and drug unit (i.e. MMAE) through linker units between them. The linker can be a peptidyl linker with a length of at least 2-3 amino acid residues, which can be optionally released by enzyme cleavage, or can be pH sensitive, or not cleavable, and the drug can be released through antibody degradation. The preferred peptidyl linker herein is Val-cit linker.

In a preferred embodiment herein, the drug portion is Doxorubicin, preferably Doxorubicin-SMCC, that is, with the linker unit being succinimidyl 4-[N-maleimidomethyl]cyclohexane-1-carboxylate (SMCC). Doxorubicin is a known commercially available drug and can also be prepared using commonly known methods.

In a preferred embodiment, Doxorubicin is preferably connected to antibody moiety via linker unit, and the preferred DAR (drug:antibody ratio) can be between 8:1 and 1:1, preferably 1, 2, 3, 4, 5, 6, 7, or 8. In the preferred embodiments of the present invention, DAR can be selected from 2, 4, or 7.

In a more preferred embodiment, Doxorubicin class ADC can link the drug portion with antibody portion (for example the preferred IgG1 antibody) via a linker unit between them, which can be a chemical linker, for example uncleavable SMCC linker (succinimidyl 4-[N-maleimidomethyl]cyclohexane-1-carboxylate), and the drug can release by the antibody degradation.

Doxorubicin-SMCC has following formula:

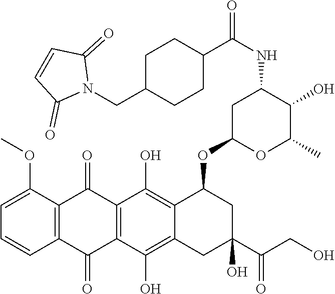 [SMILES: COc1cccc2c1C(=O)c1c(O)c3c(c(O)c1C2=O)C[C@@](O)(C(=O)CO)C[C@@H]3O[C@H]1C[C@H](NC(=O)C2CCC(CN3C(=O)C=CC3=O)CC2)[C@H](O)[C@H](C)O1]

In a preferred embodiment, the drug portion is SN-38, which is a topoisomerase I inhibitor and metabolite of irinotecan. SN-38 is a known commercially available drug in this field, and can also be prepared using commonly known methods.

In a preferred embodiment, SN38 is preferably connected to antibody moiety via linker unit, and the preferred DAR (drug:antibody ratio) can be between 8:1 and 1:1, preferably 1, 2, 3, 4, 5, 6, 7, or 8. In the preferred embodiments of the present invention, DAR can be selected from 2, 4, or 7.

In a more preferred embodiment, irinotecan class ADC can link the drug portion with antibody portion (for example the preferred IgG1 antibody) via a linker unit between them, which can be a chemical linker, for example hydrolyzable CL2A linker to release drug in vivo.

CL2A-SN38 has the following structure:

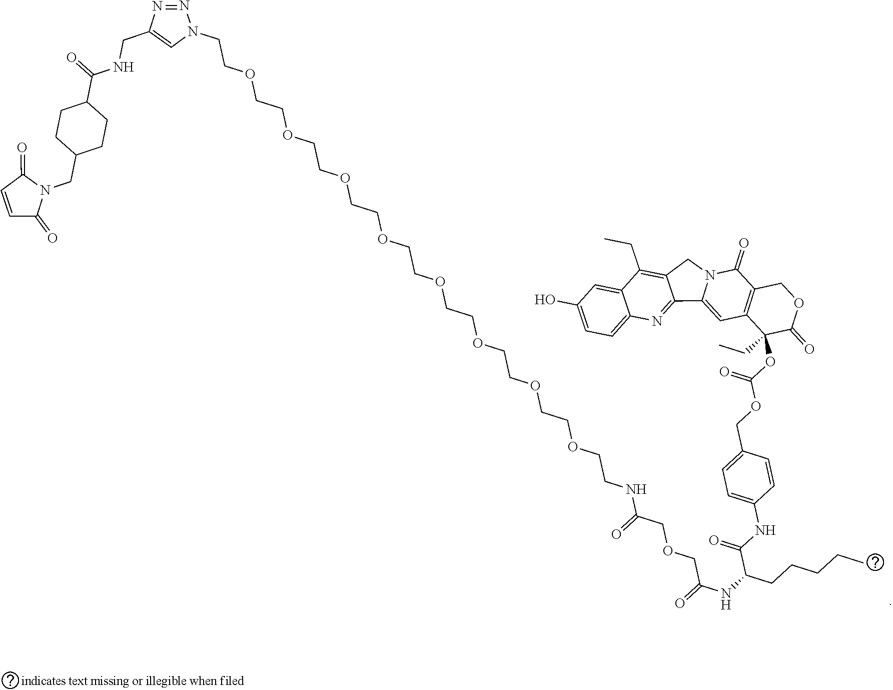 [SMILES: CCCCC[C@H](NC(=O)COCC(=O)NCCOCCOCCOCCOCCOCCOCCOCCOCCn1cc(CNC(=O)C2CCC(CN3C(=O)C=CC3=O)CC2)nn1)C(=O)Nc1ccc(COC(=O)O[C@]2(CC)C(=O)OCc3c2cc2n(c3=O)Cc3c-2nc2ccc(O)cc2c3CC)cc1]

In certain embodiments provided herein, ADC is provided in the “pharmaceutical composition”. Such pharmaceutical compositions include antibody-drug conjugates provided herein and one or more pharmaceutically acceptable or physiologically acceptable excipients. In some embodiments, antibody-drug conjugates are provided in combination with or separately from one or more other reagents. Provided herein also is a composition comprising one or more other reagents and one or more pharmaceutically acceptable or physiologically acceptable excipient(s). In particular embodiments, antibody-drug conjugates and one or more other reagents exist in therapeutically acceptable amounts. Pharmaceutical compositions can be used according to the methods and uses provided herein. The pharmaceutical composition provided herein can be formulated to be compatible with the expected administration route or method.

In some embodiments, saccharides or polyols are added as optional excipients, the saccharides including but not limited to sucrose, trehalose, lactose, starch, cellulose, and the polyols including but not limited to mannitol, sorbitol, glycerol, polyethylene glycol, etc.

The invention will be further described in the following examples, which do not limit the scope of the invention described in the claims.

Unless otherwise specifically specified, any feature, step, element, embodiment or aspect of the present invention may be combined with any other feature, step, element, embodiment or aspect. Although the present invention has been described in considerable detail with the help of description and examples for the purpose of clarification and understanding, it is obvious that certain changes and modifications can be practiced within the scope of the accompanying claims.

Unless otherwise defined, all technical and scientific terms used herein have the same meaning as commonly understood by those of ordinary skill in the relevant art. All publications and patents specifically referred to herein are incorporated by reference for all purposes.

### Example 1 Preparation of the Antibody-Drug Conjugate (ADC)

### 1. Linker Payload Information

In present invention, linker payload (MC-VC-PAB-MMAE, CL2A-SN38 and Doxorubicin-SMCC) were used for antibody conjugations, detailed information is listed in below table.

| Linker-payload ID | Vendor | CAS# |
|---|---|---|
| MC-VC-PAB-MMAE | WuXi STA(Shanghai) Co., Ltd. | 646502-53-6 |
| CL2A-SN38 | WiXi XDC | 1279680-68-0 |
| Doxorubicin-SMCC | WiXi XDC | 400647-59-8 |

The chemical structure of MC-VC-PAB-MMAE is as follows:

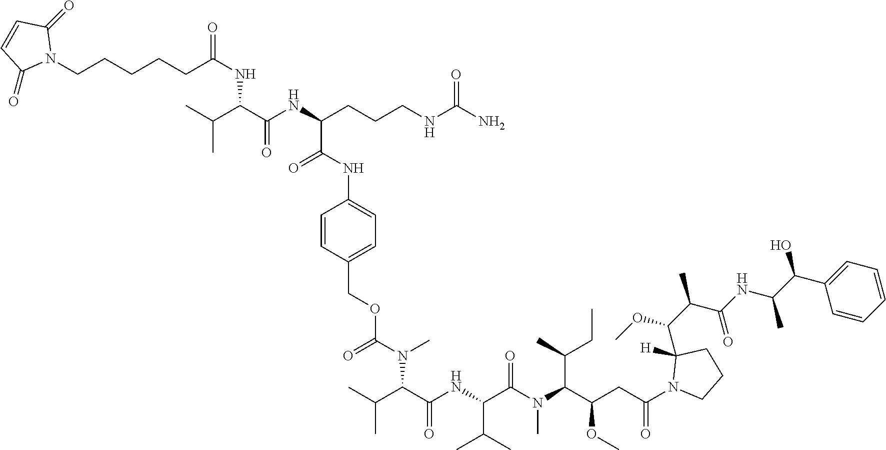 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

The chemical structure of CL2A-SN38 is illustrated below:

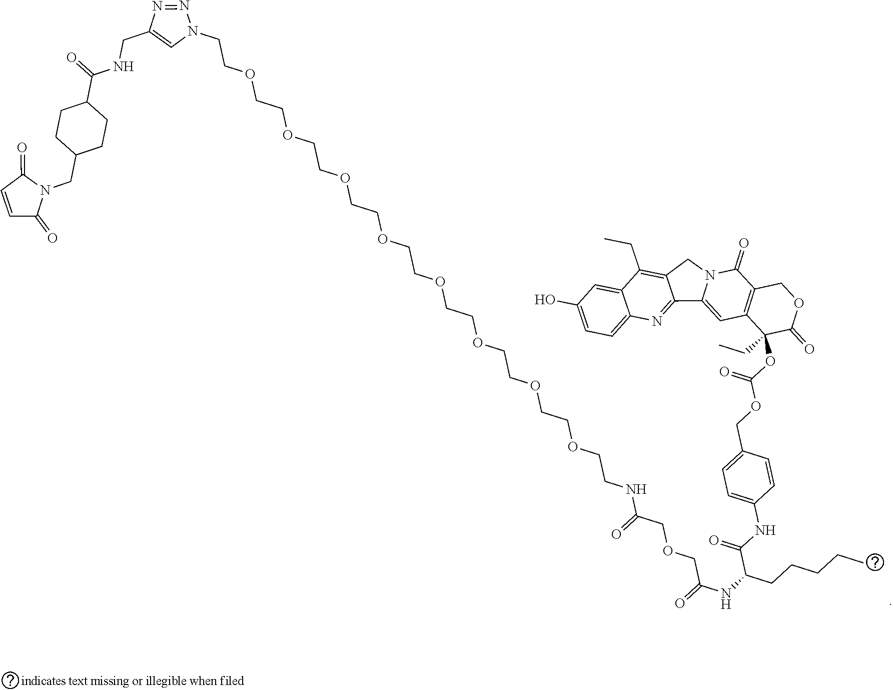 [SMILES: CCCCC[C@H](NC(=O)COCC(=O)NCCOCCOCCOCCOCCOCCOCCOCCOCCn1cc(CNC(=O)C2CCC(CN3C(=O)C=CC3=O)CC2)nn1)C(=O)Nc1ccc(COC(=O)O[C@]2(CC)C(=O)OCc3c2cc2n(c3=O)Cc3c-2nc2ccc(O)cc2c3CC)cc1]

The chemical structure of Doxorubicin-SMCC is illustrated as follows:

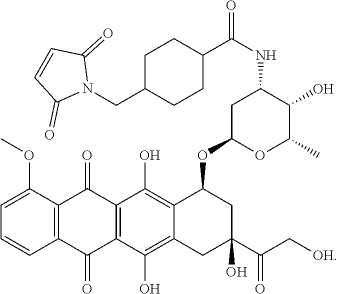 [SMILES: COc1cccc2c1C(=O)c1c(O)c3c(c(O)c1C2=O)C[C@@](O)(C(=O)CO)C[C@@H]3O[C@H]1C[C@H](NC(=O)C2CCC(CN3C(=O)C=CC3=O)CC2)[C@H](O)[C@H](C)O1]

### 2. Monoclonal Antibody Information

The monoclonal antibodies (mAbs) used for conjugation are IgG1 types expressed by CHO cell line, all the related information is listed in below Table 1:

TABLE 1
Antibody information

| Sample | | | | Molecular | Production |
|---|---|---|---|---|---|
| ID | Vendor | Target | Catalog No. | Weight | System |
| mAb-A | WuXi Biologics | HER2 | MC019-B04AD | 145.2 kDa | CHO |
| mAb-B | WuXi Biologics | Not applicable | MC016-B04T | 145.6 kDa | CHO |
| mAb-C | WuXi Biologics | INF-α | MC017-B04W | 145.2 kDa | CHO |
| mAb-D | WuXi Biologics | VEGF | MC057-C09Z | 146.3 kDa | CHO |
| mAb-E | WuXi Biologics | CD20 | MC049-C01AB | 144.2 kDa | CHO |

### 3. Conjugation Procedure and Antibody-Drug Conjugate Information

Conjugation procedure: add a certain amount of reducing agent (e.g., tris (2-carboxyethyl) phosphine) to antibody solution for reducing mAbs interchain disulfide bonds. Afterwards, calculated amounts of linker payload were added into above solutions for conjugation reactions. The reaction solution was quenched by N-Acetylcysteine and the resulted solution was stored under −70° C. condition before further usage. The drug to antibody ratio (DAR) is confirmed to be within the target ranges by using appropriate analytical methods (e.g., hydrophobic interaction chromatography). All the prepared ADCs are listed in below Table 2 for further formulation development.

TABLE 2
Summary of the ADC information:

| ADC ID | mAb ID | Linkor payload ID | DAR |
|---|---|---|---|
| ADC-A (DAR 4) | mAb-A | MC-VC-PAB-MMAE | 4 |
| ADC-B (DAR 4) | mAb-B | MC-VC-PAB-MMAE | 4 |
| ADC-C (DAR 2) | mAb-C | MC-VC-PAB-MMAE | 2 |
| ADC-C (DAR 4) | mAb-C | MC-VC-PAB-MMAE | 4 |
| ADC-C (DAR 7) | mAb-C | MC-VC-PAB-MMAE | 7 |
| ADC-D (DAR 6) | mAb-A | CL2A-SN38 | 6 |
| ADC-D (DAR 2) | mAb-A | CL2A-SN38 | 2 |
| ADC-E (DAR 6) | mAb-A | Doxorubicin-SMCC | 6 |
| ADC-E (DAR 2) | mAb-A | Doxorubicin-SMCC | 2 |
| ADC-A (DAR 6) | mAb-A | MC-VC-PAB-MMAE | 6 |
| ADC-A (DAR 2) | mAb-A | MC-VC-PAB-MMAE | 2 |
| ADC-F (DAR 2) | mAb-D | CL2A-SN38 | 2 |
| ADC-G (DAR 2) | mAb-D | Doxorubicin-SMCC | 2 |
| ADC-H (DAR 2) | mAb-E | CL2A-SN38 | 2 |
| ADC-I (DAR 2) | mAb-E | Doxorubicin-SMCC | 2 |

### Example 2. Stability Study for ADCs

### 1. ADC Formulation Preparation:

1.1 ADC substance thawing

The frozen ADC samples from Example 1 was taken out from −70° C. freezers and placed under room temperature until completely thawed.

1.2 Ultrafiltration buffer exchange

The 30 KD ultrafiltration tubes (Sartorius, VC1022) are filled with ADC-A, ADC-B, ADC-C (DAR 2), ADC-C (DAR 4) and ADC-C (DAR 7) stock solutions respectively, and weighted before and after filling the samples (ultrafiltration tube (g) and the ultrafiltration tube+ADC stock solution (g)). If the volume of the sample is smaller than the maximum fillable volume of the ultrafiltration tube, it shall be diluted to the maximum volume with 20 mM histidine buffer at pH 6.0 before the first centrifugation step, and the weight thereof will be: the ultrafiltration tube+ADC stock solution+buffer (g).

$$\begin{matrix}Buffer exchange rate = ( 1 - 1 / accumulated dilution multiple ) * 100 \% \\ Dilution multiple = ( ADC stock solution + buffer ) / ADC stock solution\end{matrix}$$

Accumulated dilution multiple=dilution multiple n*dilution multiple n+1, n=1, 2, . . . ;

Ultrafiltration and liquid exchange are carried out using centrifuge (Eppendorf, 5810R or 5804R, 4000 rpm, 5° C. and 30-60 min);

After centrifugation is completed, rediluting with 20 mM histidine buffer (pH 6.0) to the maximum volume, and weighted as ultrafiltration tube+ADC stock solution+buffer (g).

### 1.3 Repeating Ultrafiltration, Dilution and Centrifugation Until the Buffer Exchange Rate Reaches Above 98%.

### 2. Compounding and Formulation Information

According to the Table 3 below, every component of the formulation factors (excipients, polysorbate (PS) 80) was calculated and adjusted to target concentration by spiking in corresponding excipient stock solutions. Protein concentrations were adjusted to target values with corresponding buffers. 5% (w/w) polysorbate 80 stock solution is added finally to reach the target concentration in the formulation.

TABLE 3
ADC formulations used in the Example

| | | ADC | | |
|---|---|---|---|---|
| Formulation ID | ADC ID | concentration | | Formulation compositions |
| F1 | ADC-A (DAR4) | 2 | mg/mL | 20 mM histidine, 8% sucrose, 0.02% PS80, pH 6.0 |
| F2 | ADC-A (DAR4) | 2 | mg/mL | 20 mM histidine, 4% sucrose, 4% HPBCD, 0.02% PS80, pH 6.0 |
| F3 | ADC-A (DAR4) | 2 | mg/mL | 20 mM histidine, 8% HPBCD, 0.02% PS80, pH 6.0 |
| F4 | ADC-B (DAR4) | 2 | mg/mL | 20 mM histidine, 8% sucrose, 0.02% PS80, pH 6.0 |
| F5 | ADC-B (DAR4) | 2 | mg/mL | 20 mM histidine, 4% sucrose, 4% HPBCD, 0.02% PS80, pH 6.0 |
| F6 | ADC-B (DAR4) | 2 | mg/mL | 20 mM histidine, 8% HPBCD, 0.02% PS80, pH 6.0 |
| F7 | ADC-C (DAR4) | 2 | mg/mL | 20 mM histidine, 8% sucrose, 0.02% PS80, pH 6.0 |
| F8 | ADC-C (DAR4) | 2 | mg/mL | 20 mM histidine, 4% sucrose, 4% HPBCD, 0.02% PS80, pH 6.0 |
| F9 | ADC-C (DAR4) | 2 | mg/mL | 20 mM histidine, 8% HPBCD, 0.02% PS80, pH 6.0 |
| F10 | ADC-C (DAR4) | 10 | mg/mL | 20 mM histidine, 0.02% PS80, pH 6.0 |
| F11 | ADC-C (DAR4) | 10 | mg/mL | 20 mM histidine, 2% HPBCD, 0.02% PS80, pH 6.0 |
| F12 | ADC-C (DAR4) | 10 | mg/mL | 20 mM histidine, 8% HPBCD, 0.02% PS80, pH 6.0 |
| F13 | ADC-C (DAR4) | 10 | mg/mL | 20 mM histidine, 16% HPBCD, 0.02% PS80, pH 6.0 |
| F14 | ADC-C (DAR4) | 10 | mg/mL | 20 mM histidine, 8% sucrose, 0.02% PS80, pH 6.0 |
| F15 | ADC-C (DAR2) | 10 | mg/mL | 20 mM histidine, 0.02% PS80, pH 6.0 |
| F16 | ADC-C (DAR2) | 10 | mg/mL | 20 mM histidine, 8% HPBCD, 0.02% PS80, pH 6.0 |
| F17 | ADC-C (DAR2) | 10 | mg/mL | 20 mM histidine, 8% sucrose, 0.02% PS80, pH 6.0 |
| F18 | ADC-C (DAR4) | 10 | mg/mL | 20 mM histidine, 0.02% PS80, pH 6.0 |
| F19 | ADC-C (DAR4) | 10 | mg/mL | 20 mM histidine, 8% HPBCD, 0.02% PS80, pH 6.0 |
| F20 | ADC-C (DAR4) | 10 | mg/mL | 20 mM histidine, 8% sucrose, 0.02% PS80, pH 6.0 |
| F21 | ADC-C (DAR7) | 10 | mg/mL | 20 mM histidine, 0.02% PS80, pH 6.0 |
| F22 | ADC-C (DAR7) | 10 | mg/mL | 20 mM histidine, 8% HPBCD, 0.02% PS80, pH 6.0 |
| F23 | ADC-C (DAR7) | 10 | mg/mL | 20 mM histidine, 8% sucrose, 0.02% PS80, pH 6.0 |
| F24 | ADC-D (DAR 6) | 10 | mg/mL | 20 mM histidine, 8% HPBCD, 0.02% PS80, pH 6.0 |
| F25 | ADC-D (DAR 6) | 10 | mg/mL | 20 mM histidine, 8% sucrose, 0.02% PS80, pH 6.0 |
| F26 | ADC-D (DAR 2) | 10 | mg/mL | 20 mM histidine, 8% HPBCD, 0.02% PS80, pH 6.0 |
| F27 | ADC-D (DAR 2) | 10 | mg/mL | 20 mM histidine, 8% sucrose, 0.02% PS80, pH 6.0 |
| F28 | ADC-E (DAR 6) | 10 | mg/mL | 20 mM histidine, 8% HPBCD, 0.02% PS80, pH 6.0 |
| F29 | ADC-E (DAR 6) | 10 | mg/mL | 20 mM histidine, 8% sucrose, 0.02% PS80, pH 6.0 |
| F30 | ADC-E (DAR 2) | 10 | mg/mL | 20 mM histidine, 8% HPBCD, 0.02% PS80, pH 6.0 |
| F31 | ADC-E (DAR 2) | 10 | mg/mL | 20 mM histidine, 8% sucrose, 0.02% PS80, pH 6.0 |
| F32 | ADC-A (DAR 6) | 10 | mg/mL | 20 mM histidine, 8% HPBCD, 0.02% PS80, pH 6.0 |
| F33 | ADC-A (DAR 6) | 10 | mg/mL | 20 mM histidine, 8% sucrose, 0.02% PS80, pH 6.0 |
| F34 | ADC-A (DAR 2) | 10 | mg/mL | 20 mM histidine, 8% HPBCD, 0.02% PS80, pH 6.0 |
| F35 | ADC-A (DAR 2) | 10 | mg/mL | 20 mM histidine, 8% sucrose, 0.02% PS80, pH 6.0 |
| F36 | ADC-F (DAR 2) | 2 | mg/mL | 20 mM histidine, 8% HPBCD, 0.02% PS80, pH 6.0 |
| F37 | ADC-F (DAR 2) | 2 | mg/mL | 20 mM histidine, 8% sucrose, 0.02% PS80, pH 6.0 |
| F38 | ADC-G (DAR 2) | 2 | mg/mL | 20 mM histidine, 8% HPBCD, 0.02% PS80, pH 6.0 |
| F39 | ADC-G (DAR 2) | 2 | mg/mL | 20 mM histidine, 8% sucrose, 0.02% PS80, pH 6.0 |
| F40 | ADC-H (DAR 2) | 2 | mg/mL | 20 mM histidine, 8% HPBCD, 0.02% PS80, pH 6.0 |
| F41 | ADC-H (DAR 2) | 2 | mg/mL | 20 mM histidine, 8% sucrose, 0.02% PS80, pH 6.0 |
| F42 | ADC-I (DAR 2) | 2 | mg/mL | 20 mM histidine, 8% HPBCD, 0.02% PS80, pH 6.0 |
| F43 | ADC-I (DAR 2) | 2 | mg/mL | 20 mM histidine, 8% sucrose, 0.02% PS80, pH 6.0 |

HPBCD: Hydroxypropyl-β-cyclodextrin
PS80: Polysorbate 80

### 3. Filling and Capping

The prepared ADC formulation samples were aseptically filtered with 0.22 μm PVDF membrane filters before filling into vials. After filtration, a certain amount (e.g., 1-3.3 mL/bottle) samples were filled to sterile neutral borosilicate glass vials, capped and sealed. The prepared samples are stored in refrigerator (2-8° C.) for further stability study.

### 4. Sample Placement

During manufacturing, storage, transportation and usage, ADC drug products might be undergone with several stresses (thermal, agitation, light stresses, etc.) To evaluate the protection effect of HPBCD towards ADCs, several typical stresses including thermal, agitation and light were applied to the ADC drug products.

### 4.1 Placement Under High Temperature and Accelerated Conditions for Stability

After filling, the sample is placed in a stability incubator (MMM Group, CLC-BIV-M/CLC707-TV) for incubation. The stability incubator parameters are set to 25° C.±2° C./60% RH±5% RH (accelerated condition) or 40° C.±2° C./75% RH±5% RH (high temperature condition).

### 4.2 Shake Placement for Stability

Place the filled vials in constant temperature shaker (Shanghai Tiancheng, TS-80C) at 25° C. to test the stability of the sample. The shaker parameters are set to (300 rpm, 7 days).

### 4.3 Light and Ultraviolet Stability Evaluation

The filled samples were placed into a light stability box (Caron, 6545-3) to examine the light stability of the sample. The visible light parameters in the light stability box were set to be carried out firstly as visible light treatment (5000 lux visible light illumination, 10 days), then set the illumination parameters to ultraviolet light, the parameter is (4 W/(h·m²), 50 hours), and continue the ultraviolet illumination treatment for further 50 hours.

### 5. Stability Test

5.1 Sampling: Take the Sample Out of the Stability Incubator or Constant Temperature Shaker at the Set Sampling Time and Package them in a Biosafety Cabinet for Inspection by SEC-HPLC and iCIEF. Detection Methods are as Follows:
5.2 SEC-HPLC assay

SEC-HPLC is a purity assay that isolates molecules in a solution based on size. Connect SEC chromatography column (300×7.8 mm, 5 m) using Agilent high-performance liquid chromatography system (1260 series) for the assay. The sample temperature is set to 5±3° C., and the column temperature is set to 25±3° C. The mobile phase consists of 50 mM PB (phosphate buffer), 300 mM NaCl, pH 6.8±0.1, and the flow rate is set to 1.0 mL/min. Dilute the sample to a concentration of 10 mg/mL using mobile phase and 100 g sample is injected into the system (running time: 20 min, detection wavelength: 280 nm). Use Agilent CDS software for data analysis to quantitatively determine the aggregation degree of the ADCs based on peak abundance as the percentage of aggregates.

5.3 iCIEF assay

Imaged capillary isoelectric focusing (iCIEF) is a purity analysis instrument used to monitor the distribution of protein charge variants. The isoelectric point (pI) is an inherent characteristic of a specific protein, which is the pH value at which protein molecules do not carry a net charge. Under an external electric field, the charge variant moves along the continuous pH gradient formed by the amphoteric electrolyte of the carrier and stops at a pH equal to its pI. 20 μg sample and 80 μL premix buffer (consisting of a carrier amphoteric electrolyte, a pH marker, methylcellulose, and urea) are mixed. Detection was performed on a ProteinSimple iCE3 analyzer equipped with a whole-column detection capillary coated with fluorocarbon (FC), with a detection wavelength of 280 nm, to evaluate the charge variant distribution in different pI ranges.

The Chrom Perfect Analysis software was used to quantify the pI value and relative abundance of the separated peaks.

### 6. Result

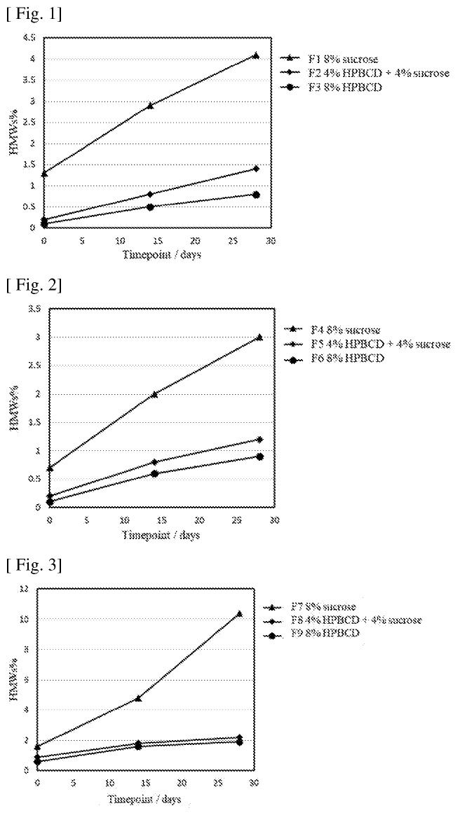

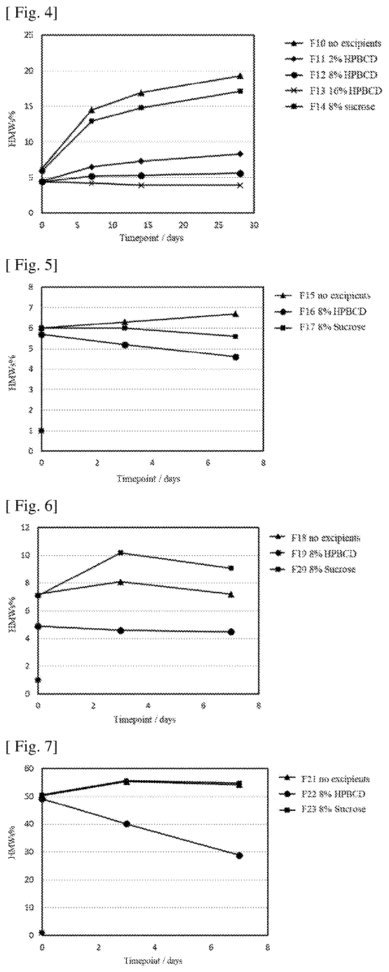

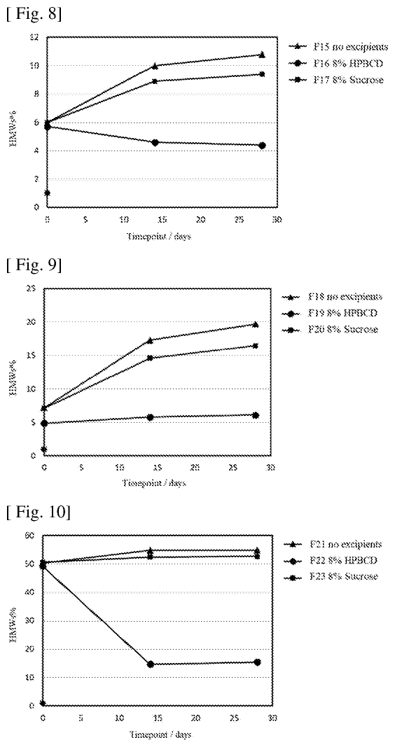

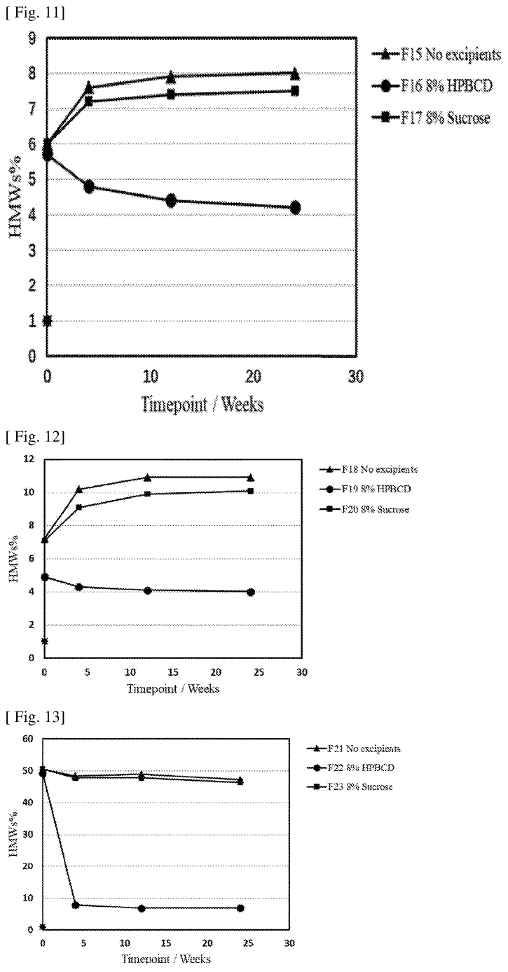

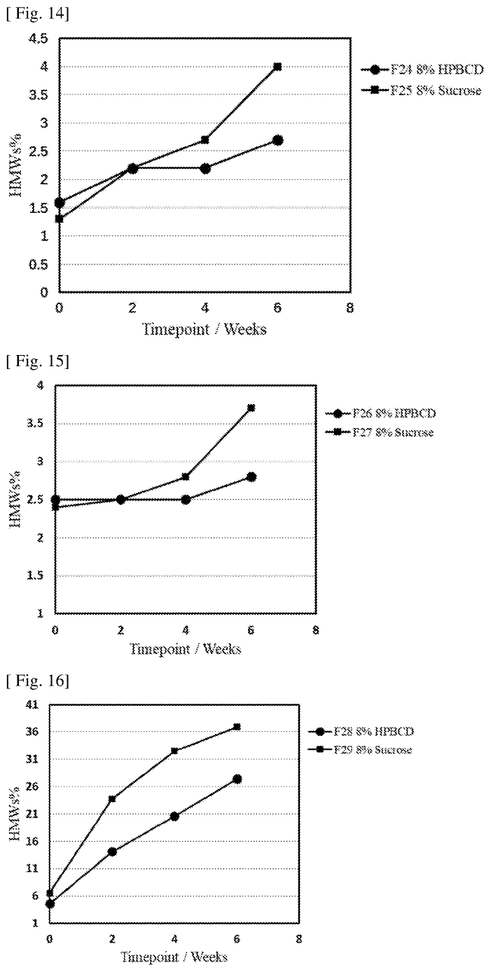

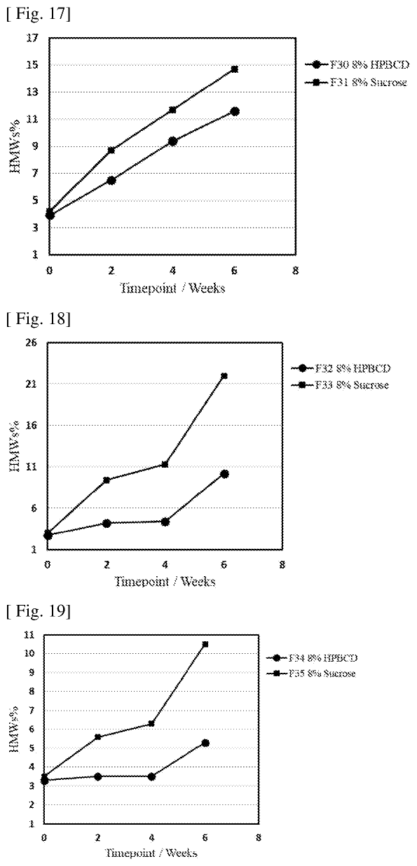

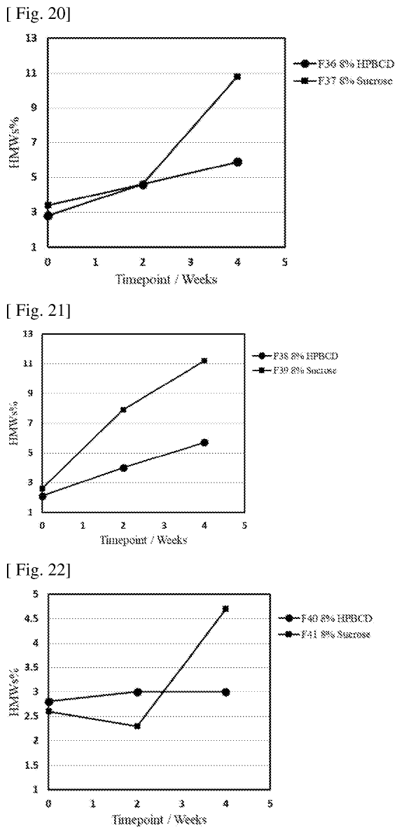

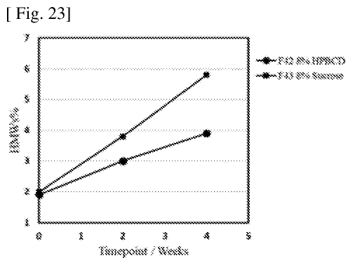

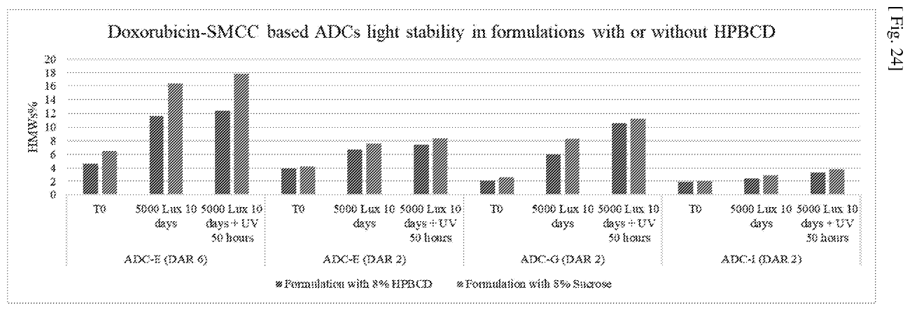

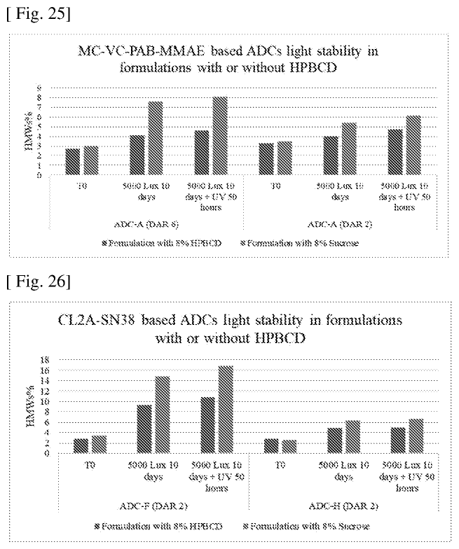

1) As shown in FIGS. 1-4, in the solution, different concentrations of HPBCD or the mixture of HPBCD and sucrose have significantly better inhibitory effect on the aggregate formation of three different kinds of ADCs (Formulations 1-14) based on IgG1 antibodies with different specificities at different DARs than using sucrose alone, indicating that the addition of HPBCD in MMAE-ADC formulations played a significant role in stabilizing.
2) As shown in FIGS. 5-7, 8-10, and 11-23, the inhibitory effects of HPBCD on the aggregate formation of ADCs with different DAR values (DAR=2, 4, and 7) in solution are significantly better than that of the commonly used excipient sucrose (8%) or without excipient (Formulations 15, 18, and 21) under different storage conditions (shaking at 25° C., high temperature and humidity at 40° C., and accelerated conditions at 25° C.).
3) As shown in FIGS. 24-26, it is confirmed that the formulations with 8% HPBCD has significantly lower increase of the content of high molecule species after light illumination and ultraviolet (UV) treatment.

In summary, compared with traditional excipients, adding hydroxypropyl-β-cyclodextrin can effectively improve the stability of the antibody-drug conjugate formulations under shaking, stressed high temperature, accelerated temperature or light/UV stress conditions, thus bringing great convenience to the storage and transportation of ADC formulations.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

[1] Adem, Y. T., Schwarz, K. A., Duenas, E., Patapoff, T. W., Galush, W. J., &Esue, O. (2014). Auristatin antibody drug conjugate physical instability and the role of drug payload. Bioconjugate Chemistry, 25 (4), 656-664. https://doi.org/10.1021/bc400 439x
[2] Ross, P. L., &Wolfe, J. L. (2016). Physical and Chemical Stability of Antibody Drug Conjugates: Current Status. Journal of Pharmaceutical Sciences, 105 (2), 391-397. https://doi. org/10.1016/j. xphs. 2015.11.037

1. A composition for stabilizing antibody-drug conjugate (ADC) formulations, characterized in that the composition comprises:
hydroxypropyl-β-cyclodextrin of about 2% to 16% (w/v), preferably of about 2%, 4%, or 8% (w/v), more preferably 8% (w/v);
optionally 0-10% (w/v) saccharides or polyols, preferably sucrose;
surfactants; and
buffering solution;
wherein the pH of the composition is in the range of 4.5-8.0, preferably 5.5-6.5 or 5.7-6.3.

2. The composition as claimed in claim 1, characterized in that the composition comprises 0-8% (w/v), preferably 4% (w/v) sucrose, and more preferably no sucrose.

3. The composition as claimed in claim 1, characterized in that the composition comprises 8% (w/v) hydroxypropyl-β-cyclodextrin and 0% (w/v) sucrose.

4. The composition as claimed in claim 1, characterized in that the composition further comprises 20 mM histidine, and wherein the pH of the composition is 6.0; wherein the surfactant is polysorbate 80, preferably of about 0.02% (w/v).

5. The composition as claimed in claim 1, wherein the drug portion of the antibody-drug conjugate (ADC) comprises a drug selected from the group consisted of Monomethyl auristatin E (MMAE), Doxorubicin, or SN-38, preferably a drug selected from MC-Val-Cit-PAB-MMAE, CL2A-SN38 (SN-38 connected by CL2A linker), or Doxorubicin-succinimidyl 4-[N-maleimidomethyl]cyclohexane-1-carboxylate (SMCC), and wherein the antibody portion of ADC is preferably a monoclonal antibody (mAb), more preferably selected from the group consisted of monoclonal antibodies targeting against tumor necrosis factor α (TNF-α), monoclonal antibodies targeting against Vascular endothelial growth factor (VEGF), monoclonal antibodies targeting against cluster of differentiation 20 (CD20), or monoclonal antibodies targeting against human epidermal growth factor receptor 2 (HER2); more preferably the antibody portion is IgG isotype, even more preferably, the IgG1 or IgG4 isotype.

6. Use of hydroxypropyl-β-cyclodextrin for stabilizing antibody-drug conjugate (ADC) formulation, characterized in that the formulation comprises hydroxypropyl-β-cyclodextrin of about 2% to 16% (w/v), preferably of about 2%, 4%, or 8% (w/v), more preferably 8% (w/v);
optionally 0-10% (w/v) saccharides or polyols, preferably sucrose;
surfactants; and
buffering solution;
wherein the pH of the formulation is in the range of 4.5-8.0, preferably 5.5-6.5 or 5.7-6.3; most preferably 5.5 or 6.0.

7. The use as claimed in claim 6, wherein the formulation comprises antibody-drug conjugate, wherein the drug portion of the antibody-drug conjugate (ADC) comprises a drug selected from the group consisted of Monomethyl auristatin E (MMAE), Doxorubicin, or SN-38, preferably a drug selected from MC-Val-Cit-PAB-MMAE, CL2A-SN38 (SN-38 connected by CL2A linker), or Doxorubicin-succinimidyl 4-[N-maleimidomethyl]cyclohexane-1-carboxylate (SMCC), and wherein the antibody portion of ADC is preferably a monoclonal antibody (mAb), more preferably selected from the group consisted of monoclonal antibodies targeting against tumor necrosis factor α (TNF-α), monoclonal antibodies targeting against Vascular endothelial growth factor (VEGF), monoclonal antibodies targeting against cluster of differentiation 20 (CD20), or monoclonal antibodies targeting against human epidermal growth factor receptor 2 (HER2); more preferably the antibody portion is IgG isotype, even more preferably, the IgG1 or IgG4 isotype, preferably, wherein the formulation comprises 8% (w/v) hydroxypropyl-β-cyclodextrin and 0% (w/v) sucrose, more preferably the formulation further comprises 20 mM histidine, and wherein the pH of the formulation is 6.0; wherein the surfactant is polysorbate 80 and preferably of about 0.02% (w/v).

8. The use as claimed in claim 6, characterized in that the Monomethyl auristatin E (MMAE) is MC-Val-Cit-PAB-MMAE, of following formula:

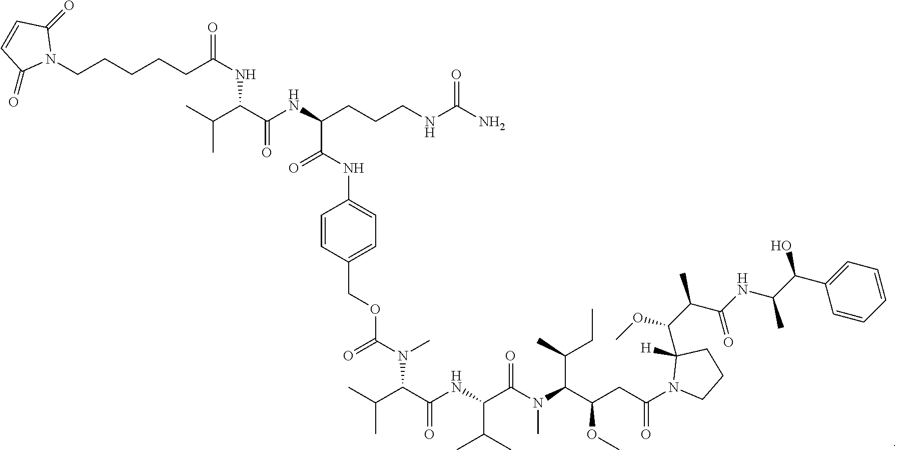 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

9. The use as claimed in claim 8, characterized in that the antibody is an IgG isotype, preferably IgG1 or IgG4; more preferably, the antibody is conjugated with MMAE via interchain cysteine; wherein the preferred MMAE:antibody ratio is between 8:1 and 1:1, with preferred values of 1, 2, 3, 4, 5, 6, 7, and 8, more preferable that DAR is selected from 2, 4, or 7.

10. The use as claimed in claim 6, characterized in that the formulation comprises 2 mg/ml to 20 mg/ml of ADC, preferably 10 mg/ml of ADC.

11. The use as claimed in claim 6, characterized in that the SMCC is Doxorubicin-succinimidyl 4-[N-maleimidomethyl]cyclohexane-1-carboxylate (SMCC), with the following formula:

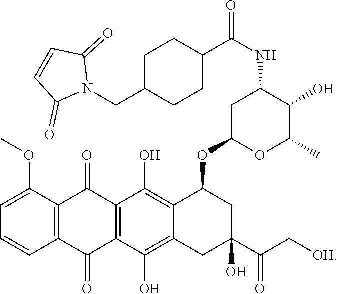 [SMILES: COc1cccc2c1C(=O)c1c(O)c3c(c(O)c1C2=O)C[C@@](O)(C(=O)CO)C[C@@H]3O[C@H]1C[C@H](NC(=O)C2CCC(CN3C(=O)C=CC3=O)CC2)[C@H](O)[C@H](C)O1]

12. The use as claimed in claim 11, characterized in that the antibody is an IgG isotype, preferably IgG1 or IgG4; more preferably, the antibody is conjugated with SMCC via interchain cysteine; wherein the preferred MMAE:antibody ratio is between 8:1 and 1:1, with preferred values of 1, 2, 3, 4, 5, 6, 7, and 8, more preferable that DAR is selected from 2, 4, or 7.

13. The use as claimed in claim 11, characterized in that the formulation comprises 2 mg/ml to 20 mg/ml of ADC, preferably 10 mg/ml of ADC

14. The use as claimed in claim 6, characterized in that the SN-38 is CL2A-SN38 (SN-38 linked by CL2A linker), having the following formula:

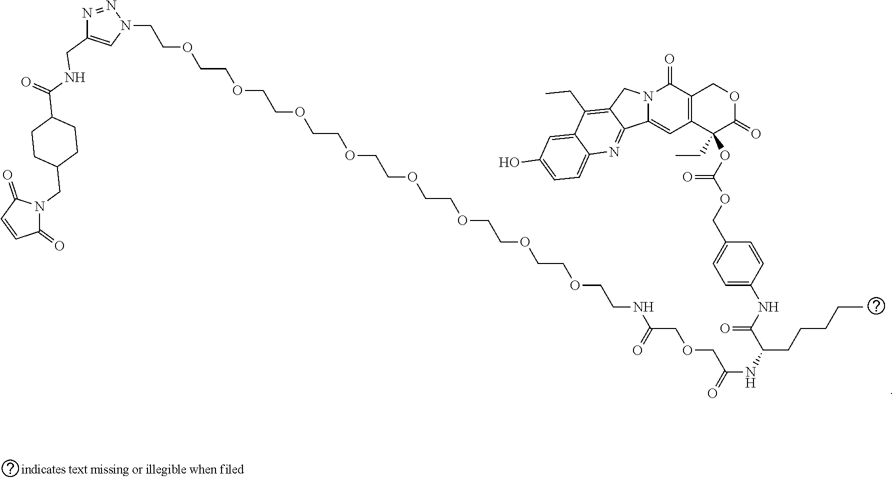 [SMILES: CCCCC[C@H](NC(=O)COCC(=O)NCCOCCOCCOCCOCCOCCOCCOCCOCCn1cc(CNC(=O)C2CCC(CN3C(=O)C=CC3=O)CC2)nn1)C(=O)Nc1ccc(COC(=O)O[C@]2(CC)C(=O)OCc3c2cc2n(c3=O)Cc3c-2nc2ccc(O)cc2c3CC)cc1]

15. The use as claimed in claim 14, characterized in that the antibody is an IgG isotype, preferably IgG1 or IgG4; more preferably, the antibody is conjugated with SN38 via interchain cysteine; wherein the preferred SN38:antibody ratio is between 8:1 and 1:1, with preferred values of 1, 2, 3, 4, 5, 6, 7, and 8, more preferable that DAR is selected from 2, 4, or 7.

16. The use as claimed in claim 14, characterized in that the formulation comprises 2 mg/ml to 20 mg/ml of ADC, preferably 10 mg/ml of ADC.
